SEQUENCE LISTING (3 sequences)

SEQ ID NO: 1 - DNA, 12 bp, Human
gttacagcacag

SEQ ID NO: 2 - DNA, 17 bp, Human
atgctgcccggtttggc

SEQ ID NO: 3 - DNA, 19 bp, Human
ctagttctgcatctgctca

(19) United States
(12) Patent Application Publication
(10) Pub. No.: US 2006/0088910 A1
(43) Pub. Date: Apr. 27, 2006

(54) RT-PCR-based cloning of the human beta-amyloid precursor protein gene and the construction of its expression plasmids

(76) Inventor: Khue Vu Nguyen, San Diego, CA (US)

Correspondence Address: H-Mai T. Nguyen, Ph.D., 2120-C Las Palmas Drive, Carlsbad, CA, 92009, US

(21) Appl. No.: 10/777,592

(22) Filed: Feb. 13, 2004

(51) Int. Cl. C12P 21/06; C12P 19/34; C12N 15/74

(52) U.S. Cl. 435/69.1; 435/91.2; 435/473

(57) ABSTRACT

Beta-amyloid peptide (βA) is a major fibrillar component of neuritic plaques in Alzheimer's disease brains and is related to the pathogenesis of the disease. βA generation depends on proteolytic cleavage of the amyloid precursor protein (APP). The present invention is a new procedure for the cloning of human βA precursor protein gene (human APP gene) based on the reverse transcription (RT) and the polymerase chain reaction (PCR). This procedure for cloning human APP gene by means of RT-PCR reactions is cost-effective, not time-consuming, and is suited for any laboratory. The present invention also includes a new procedure for the construction of expression plasmids, a/ using the pFastBac™ HTb and the pBlueBacHis2 A transfer vectors for the purpose of obtaining human APP in insect cells; and b/ using the pET-28a (+) transfer vector for the purpose of obtaining human APP in bacteria. The present invention makes it easier to obtain full-length APP which is essential for the identification of biological activities that occur in the APP molecule and for the identification of proteases capable of creating βA. Knowing which protease creates βA is important for the exploration of therapeutic and preventative strategies for the treatment of Alzheimer's disease.

### FIELD OF THE INVENTION

Beta-amyloid peptide (βA) is a major fibrillar component of neuritic plaques in Alzheimer's disease (AD) brains and is related to the pathogenesis of the disease. βA generation depends on proteolytic cleavage of the amyloid precursor protein (APP). In order to elucidate the mechanism of the inappropriate processing that leads to βA formation in Alzheimer's disease, a simple procedure suited for any laboratory to produce full-length APP is essential. The present invention is a new procedure to produce full-length APP: The cloning of human βA precursor protein gene (human APP gene) based on the reverse transcription (RT) and the polymerase chain reaction (PCR), and the construction of the expression plasmids in order to obtain full-length human APP.

### BACKGROUND OF THE INVENTION

Alzheimer's disease (AD) is a progressive neurodegenerative disease of elderly, characterized by memory loss and dementia. It is the most frequent cause of dementia in the elderly, accounting for more than 15 millions cases worldwide. It is pathologically characterized by proteinaceous deposits in various areas of the brain, particularly in the hippocampus and cerebral cortex (1, 2). Such deposits include extracellular in the form of amyloid plaques and cerebrovascular amyloid as well as intracellular in the form of neurofibrillary tangles comprising tau protein filaments. Ultrastructurally, amyloid plaques and cerebrovascular amyloid contain 6-10 nm straight filaments, which are comprised of a 42-43 amino acid subunit, the so called beta-amyloid peptide (βA) (1, 2).

The βA, a 4-kDa peptide of 39-43 amino acids, is a metabolic product of a large transmembrane amino acid precursor molecule called the amyloid precursor protein (APP). APP has several isoforms generated by alternative splicing of a pre-mRNA transcribed from a single 19 exon gene located on the long arm of chromosome 21 (3-5). Three alternatively spliced forms of APP that contain the βA sequences have been identified; they are characterized as the 695-amino acid form (APP₆₉₅) (6), and two longer forms of 751 (APP₇₅₁) (7,8) and 770 (APP₇₇₀) (9) amino acids. Each of these two latter forms contains an extra exon which encodes a Kunitz-type serine protease inhibitor (KPI) domain. The secreted version of APP₇₅₁ has been shown to be identical to protease nexin II (10, 11), and to serve as an inhibitor of coagulation factor XI (12). The secreted form of APP and fragments of APP have been implicated in a variety of other biological functions such as mitogenesis, neurotoxicity, cell adhesion, and neurotrophism (13-17).

Normally, APP is efficiently cleaved and secreted from a variety of cell types (18-20). The normal cleavage and constitutive secretion cannot lead to βA deposition (21). The formation of βA is therefore considered to be due to abnormal processing of the APP. The mechanism of this abnormal processing is unknown. Certain cases of inherited AD have been shown to result from mutations in APP that lead to enhanced cellular production of the amyloidotic βA₁₋₄₂ peptide which is less soluble and more amyloidogenic than the more common 40-amino acid species (22). The process of soluble amyloid aggregation into insoluble fibrils is associated with neurotoxicity (23), although it is not entirely clear how this is mediated. Furthermore, transgenic mice overexpressing the Lys⁶⁷⁰-Asn, Met⁶⁷¹-Leu double mutation in the APP gene have been shown to have age-related AD-like cognitive changes, amyloid plaques, raised levels of the 40-amino acid form of βA and even greater elevations of the 42-43 amino acid form of βA (24). Therefore, an over-abundance of βA and its accumulation as amyloid fibrils trigger disease pathology. Thus, the above-mentioned experiments' results provide convincing evidence that APP abnormalities can cause AD. Fundamental and important questions regarding the biological function of APP and the mechanism of abnormal processing of APP that leads to βA formation in Alzheimer's disease remain unanswered. Thus, obtaining full-length APP is essential for the identification of biological activities that occur in APP and for the identification of proteases capable of creating βA. Knowing which protease creates βA is important for the exploration of therapeutic and preventative strategies toward the treatment of Alzheimer's disease. Currently, the procedure to obtain full-length APP is the isolation of APP-cDNA clones from the cDNA library using the synthesized oligonucleotide probes; this method of probing in order to identify appropriate APP-cDNA for cloning is costly, very time consuming (requiring labor intensive steps) and requires highly skilled personnel. Consequently, very few laboratories have the equipment or capability to produce full-length APP; for that reason, other laboratories, which do not have such capability, would have to obtain APP-cDNA clones from the few laboratories that can produce APP-cDNA clones. There is therefore a need to design an easy, simple and cost-effective procedure to obtain APP-cDNA for cloning. For such a purpose, in the present invention, a different approach is used: Instead of using probes to isolate APP-cDNA clones, the new procedure involves starting with the human APP-messenger ribonucleic acid (APP-mRNA) and utilizing the RT and PCR reactions in order to obtain APP-cDNA for closing. This is a simple and cost-effective procedure that can be performed in any laboratory.

In addition, the cloning of APP-cDNA based on RT-PCR reactions allows the construction of expression plasmids, (a) using the pFastBac™ HTb and the pBlueBacHis2 A transfer vectors for the purpose of obtaining human APP in insect cells; and (b) using the pET-28a (+) transfer vector for the purpose of obtaining human APP in bacteria.

### PURPOSE OF THE INVENTION

The purpose of the invention is: (1) To perform the procedure to obtain APP-cDNA for cloning by starting with APP-messenger ribonucleic acid (APP-mRNA) and by utilizing the RT and PCR reactions; and (2) to construct expression plasmids, (a) using the pFastBac™ HTb and the pBlueBacHis2 A transfer vectors for the purpose of obtaining human APP in insect cells; and (b) using the pET-28a (+) transfer vector for the purpose of obtaining human APP in bacteria. The availability of full-length APP is essential for the identification of biological activities that occur in APP and of proteases capable of creating βA in Alzheimer's disease. Compounds effective in inhibiting the βA formation could serve as potential therapeutic agents in the future for the treatment of Alzheimer's disease.

### MATERIALS AND METHODS

Isolation of RNA

The total ribonucleic acid (RNA) was isolated from a sample of human liver biopsy (wild type) according to the method described by Sambrook et al. (25) using guanidin/phenol (Tris Reagent™, Euromedex, 67460 Souffelweyersheim, France). The total RNA was dissolved in water pre-treated by 0.1% diethyl pyrocarbonate (DEPC, Sigma, St. Louis, Mo.). This RNA solution is ready for subsequent treatment for the synthesis of the cDNA.

Reverse Transcription

The synthesis of the cDNA was performed by reverse transcription (RT), as described by Sambrook et al. (25). The first copies of cDNA were synthesized by using the synthesized oligonucleotide (SEQ ID NO. 1) (Genosys Biotechnologies, Europe, Ltd., France) with the following sequence: 5′ GTTACAGCACAG 3′ (SEQ ID NO. 1). This oligonucleotide was selected by taking the complementary sequence between base pairs 2,367 and 2,378 of the human mRNA for APP₆₉₅ described by Kang et al. (6) (GenBank Accession No. Y00264). The M-MLV Reverse Transcriptase enzyme (Gibco BRL^{R}, Life Technologies Sarl, BP 96, 95613 Cergy Pontoise, France) was used to perform the reverse transcription reaction. This reaction was performed as follow:

0.1 nmol of oligonucleotide SEQ ID NO. 1, and 10 pmol each of nucleotides dATP, dCTP, dGTP, and dTTP were added to 5 μg of total RNA. The reaction was conducted in the presence of a reaction buffer for RT of the Gibco BRL^{R} kit; the total volume of the reaction was 20 μl. After heating the mixture to 90° C. for 2 minutes and then cooling it on ice for 1 minute, 200 U M-MLV were added; then the mixture was left to 25° C. for 10 minutes and then to 42° C. for 45 minutes.

Amplification

Having obtained the first copies of cDNA by RT reaction, the next step is to amplify the RT products.

Amplifying the RT products were assessed by using the polymerase chain reaction (PCR) technique (26, 27). Two synthesized oligonucleotides (SEQ ID NO. 2) (forward primer) and (SEQ ID NO. 3) (reverse primer) (Genosys Biotechnologies) were used in order to perform the PCR reaction. They have the following sequences:
5′ ATGCTGCCCGGTTTGGC 3′ (SEQ ID NO. 2) and
5′CTAGTTCTGCATCTGCTCA 3′ (SEQ ID NO. 3).

The oligonucleotide SEQ ID NO. 2 was based on the sequence between base pairs 148 and 164 of the human mRNA sequence for APP₆₉₅ described by Kang et al. (6) (GenBank Accession No. Y00264). To allow PCR, the oligonucleotide SEQ ID NO. 3 was selected by taking the complementary sequence between base pairs 2,217 and 2,235 of the human mRNA sequence for APP₆₉₅ described by Kang et al. (6) (GenBank Accession No. Y00264). Amplification was conducted by using a DNA Thermal Cycler (Amplitron^{R} II Thermolyne). The reaction was conducted in a total volume of 50 μl with 2.5 U of Taq DNA polymerase (Promega Corporation, Madison, Wis., U.S.A.) in the presence of the PCR reaction buffer from Promega kit containing 0.1 nmol of oligonucleotide (SEQ ID NO. 2) and 0.1 nmol of oligonucleotide (SEQ ID NO. 3), 10 pmol each of nucleotides dATP, dCTP, dGTP, and dTTP, 62.5 pmol of MgCl₂ (Promega) and 5 μl of the reverse transcription reaction medium obtained previously. Amplification conditions were as follow: Denaturating at 94° C. for 1 minute, annealing at 55° C. for 2 minutes, and elongating at 72° C. for 3 minutes, each for 35 cycles. The PCR product was analyzed by electrophoresis on a 20 g/l agarose gel to screen for the presence of the appropriate-size band by using the fluorescent dye ethidium bromide. The PCR product was then isolated, purified by phenol-chloroform extraction, dried and resuspended in distilled water according to the method described by Sambrook et al. (25).

Cloning

The obtained purified PCR product was then subjected to the ligation reaction into the pCR^{R} II plasmid vector of the TA Cloning kit (Invitrogen). The reagents of this kit and the reaction conditions performed according to the manufacturer's recommendations were used. The ligation product was then introduced in INVαF′ E. Coli strain by using the reagents and the transformation procedure of the TA Cloning kit (Invitrogen). The screening for inserts was performed by using blue-white color selection. The sequencing of inserts obtained was performed by using the ABI DNA sequencer. The resulting vectors were termed (1) (pCR^{R} II/APP₇₅₁-cDNA) and (2) (pCR^{R} II/APP₇₇₀-cDNA).

Construction of the Expression Plasmids for Human APP

To construct the expression plasmids for human APP, the following systems were performed:

1. Insect Expression Systems

1.1. Using the Bac-to-Bac^{R} Baculovirus Expression System

From the vectors (1) and (2) obtained previously, the XbaI to HindIII fragments containing the cDNA coding sequences of APP were isolated and subjected to the ligation reaction into the baculovirus transfer vectors pFastBac™ HTb (Invitrogen) predigested by XbaI and HindIII and predephosphorylated with calf intestinal alkaline phosphatase (Boehringer Mannheim, GmbH, Germany). The reaction was conducted in the presence of the reagents for the ligation of the TA Cloning kit (Invitrogen); the reaction conditions used are according to the manufacturer's recommendations. The ligation products were then introduced in INVαF′ E. Coli strain (Invitrogen) by using the reagents and the transformation procedure of the TA Cloning kit (Invitrogen). The screening for inserts was based on the presence of white colonies. The resulting vectors thus obtained were termed (3) for pFastBac™ HTb containing APP₇₅₁-cDNA (pFastBac™ HTb/APP₇₅₁-cDNA), and (4) for pFastBac™ HTb containing APP₇₇₀-cDNA (pFastBac™ HTb/APP₇₇₀-cDNA) respectively. The vectors (3) and (4) were then introduced in the DH10Bac™ E. Coli competent cells (Invitrogen) by using the reagents and the transformation procedure of the Bac-to-Bac^{R} Baculovirus Expression System (Invitrogen). The screening for recombinant bacmids was performed by using blue-white color selection. The verification of the presence of APP cDNA's insert in the recombinant bacmid was performed by PCR amplification using the M13 forward (−40) and M13 reverse primers (Invitrogen). The reaction conditions used are according to the manufacturer's recommendations. The PCR product was analyzed by electrophoresis on a 20 g/l agarose gel to screen for the presence of the appropriate-size band by using the fluorescent dye ethidium bromide. The PCR product was then isolated, and purified by phenol-chloroform extraction, dried and resuspended in distilled water according to the method described by Sambrook et al. (25). The sequencing of APP-cDNA's insert was performed by using the ABI DNA sequencer. The recombinant bacmids thus obtained were termed (5) for vector (3) in DH10Bac™ E. Coli and (6) for vector (4) in DH10Bac™ E. Coli respectively.

1.2. Using the Bac-N-Bac™ Baculovirus Expression System

From the vectors (3) and (4) obtained previously, the NcoI to HindIII fragments containing the cDNA coding sequences of APP were isolated and subjected to the ligation reaction into the baculovirus transfer vectors pBlueBacHis2 A (Invitrogen) predigested by NcoI and HindIII and predephosphorylated with calf intestinal alkaline phosphatase (Boehringer Mannheim, GmbH, Germany). The reaction was conducted in the presence of the reagents for the ligation of the TA Cloning kit (Invitrogen); the reaction conditions used are according to the manufacturer's recommendations. The ligation products were then introduced in the INVαF′ E. Coli strain (Invitrogen) by using the reagents and the transformation procedure of the TA Cloning kit (Invitrogen). The screening for inserts was performed by using blue-white color selection. The resulting vectors thus obtained were termed (7) for pBlueBacHis2 A containing APP₇₅₁-cDNA (pBlueBacHis2 A/APP₇₅₁-cDNA), and (8) for pBlueBacHis2 A containing APP₇₇₀-cDNA (pBlueBacHis2 A/APP₇₇₀-cDNA) respectively.

2. Prokaryotic Expression Systems

From the vectors (3) and (4) obtained previously, the SalI to HindIII fragments containing the cDNA coding sequences of APP were isolated and subjected to the ligation reaction into the pET-28a (+) plasmid vectors (Novagen) predigested by SalI and HindIII and predephosphorylated with calf intestinal alkaline phosphatase (Boehringer Mannheim, GmbH, Germany). The reaction was conducted in the presence of the reagents for the ligation of the TA Cloning kit (Invitrogen); the reaction conditions used are according to the manufacturer's recommendations. The ligation products were then introduced in the INVαF′ E. Coli strain (Invitrogen) by using the reagents and the transformation procedure of the TA Cloning kit (Invitrogen). The screening for inserts was based on the presence of white colonies. The resulting vectors thus obtained were termed (9) for pET-28a (+) containing APP₇₅₁-cDNA (pET-28a (+)/APP₇₅₁-cDNA), and (10) for pET-28a (+) containing APP₇₇₀-cDNA (pET-28a (+)/APP₇₇₀-cDNA) respectively.

### RESULTS AND DISCUSSION

Cloning of Human APP Gene

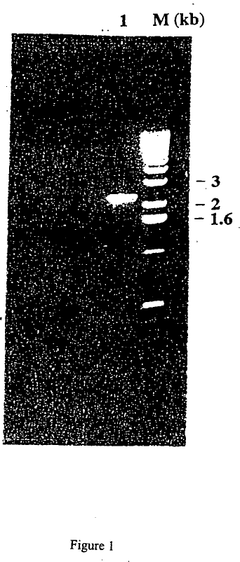

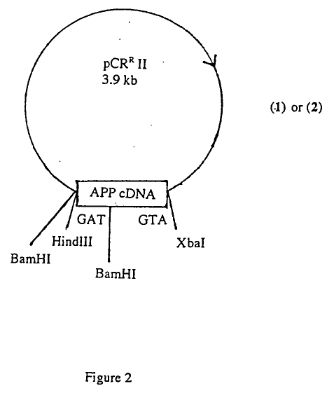

In order to investigate the molecular basis for βA deposition in Alzheimer's disease, intense efforts have been directed toward the identification of APP-cDNA clones which encode the amino acid sequences of human APP (3-9). The availability of the APP-cDNA clones has permitted the construction of the expression plasmids for human APP recombinant proteins. Expression of the human APP recombinant proteins has been performed in a variety of cell types (18-20). Up to now, the only approach to identify APP-cDNA clones from the human brain cDNA library is by using the synthesized oligonucleotide probes corresponding to the first 20 amino acids of the amino acid sequence of the βA (1, 2); however, this approach is costly, time consuming (requiring labor intensive steps) and it requires highly skilled personnel. Furthermore, not many laboratories have the equipment or capability to perform such a procedure. In contrast, the present invention is the development of a procedure that is easy, simple and cost-effective which can be performed in any laboratory; this procedure starts with the human APP-mRNA and includes the utilization of the RT and PCR reactions to obtain APP-cDNA for cloning. As shown in FIG. 1, the PCR product of ˜2.3 kb of the human APP gene was successfully amplified by two synthesized oligonucleotides (SEQ ID NO. 2) and (SEQ ID NO. 3). The obtained PCR product was subcloned into the pCR^{R} II plasmid vector of 3.9 kb (Invitrogen). The analysis of the sequence of inserts from the ten clones showed that the DNA sequences of the PCR product completely matched with the sequences of APP₇₅₁-mRNA (6 clones) (7, 8) (GenBank Accession No. Y00297) and APP₇₇₀, mRNA (4 clones) (9) (GenBank Accession No. NM_{—}000484). These results suggest that in normal human liver, the amounts of APP-mRNAs with the Kunitz-type serine protease inhibitor (KPI) domain (APP₇₅₁-mRNA and APP₇₇₀-mRNA) are predominant. Next, in order to determine the orientation of APP cDNA's insert in the plasmid vector, we chose a restriction enzyme present in both APP-cDNA and plasmid vector. This enzyme generates different sizes of DNA fragments depending on the insert's orientation. The orientation of APP-cDNA's insert in the plasmid vectors (1) (pCR^{R} II/APP₇₅₁-cDNA) and (2) (pCR^{R} II/APP₇₇₀-cDNA) (FIG. 2) was determined by the digestion of (1) and (2) with BamHI; the results show two DNA fragments of 5,393 and 760 bp for (1) and 5,450 and 760 bp for (2) (data not shown).

Construction of the Expression Plasmids for Human APP

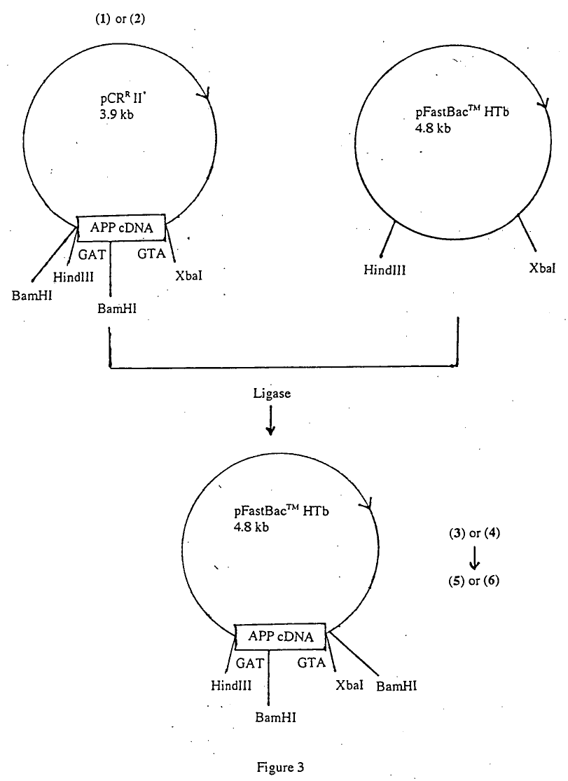

A schematic representation of the different cloning steps for constructing the expression plasmids for human APP recombinant proteins are given in FIGS. 3-5.

1. Insect Expression Systems

Insect expression systems are relative newcomers to the gene expression arena. As higher eukaryotes, insect cells offer many mammalian posttranslational modifications. Insect cells have proven to be an excellent host for recombinant protein expression. They are often chosen for protein production because they are a higher eukaryotic with easy cell culture and can be readily adapted to high-density suspension culture for large-scale expression.

1.1. Using the Bac-to-Bac^{R} Baculovirus Expression System

The Bac-to-Bac^{R} Baculovirus Expression system is the fastest route available for producing recombinant baculovirus. This system uses a unique bacmid shuttle vector that combines with an expression cassette by site-specific transposition to create an expression bacmid. The expression bacmid is then transfected into insect cells to generate high-titer, ready-to-use recombinant baculovirus. With Bac-to-Bac^{R}, recombinant protein can be expressed in as little as nine days. The Bac-to-Bac^{R} HT baculovirus expression system (Invitrogen) is used. This system offers the components necessary for expression and purification of histidine tagged recombinant proteins in Sf9, Sf21, or High Five™ cells. The system includes the pFastBac™ HTb vector of 4.8 kb, the DH10Bac™ E. Coli competent cells for cloning. Expression of the recombinant fusion proteins from the pFastBac™ HTb vector is driven by the polyhedrin promotor. Proteins expressed are fused at the N-terminus to a tag of six tandem histidine residues and a TEV protease cleavage site for removal of histidine tag following protein purification. The histidine residues create a high-affinity metal binding site to allow purification of recombinant fusion proteins on nickel-chelating resin. In the present work, a schematic representation of the different cloning steps for constructing the expression plasmids for APP using the pFastBac™ HTb vector is given in FIG. 3. The correct orientation for APP expression of the APP cDNA's insert in the plasmid vectors (3) and (4) was confirmed by the digestion of (3) and (4) with BamHI which gave two DNA fragments of 5,557 and 1,496 bp for (3) and 5,560 and 1,550 bp for (4) (data not shown). The verification of the presence of APP-cDNA's insert in the recombinant bacmids (5) and (6) using PCR technique (in the presence of M13 forward (−40) and M13 reverse primers) gave a DNA fragment of 4,683 bp for (5) and 4,740 bp for (6) respectively (data not shown). The DNA sequences of the obtained PCR product completely matched with the sequences of APP₇₅₁-mRNA (7, 8) (GenBank Accession No. Y00297) and APP₇₇₀-mRNA (9) (GenBank Accession No. NM_{—}000484).

1.2. Using the Bac-N-Bac™ Baculovirus Expression System

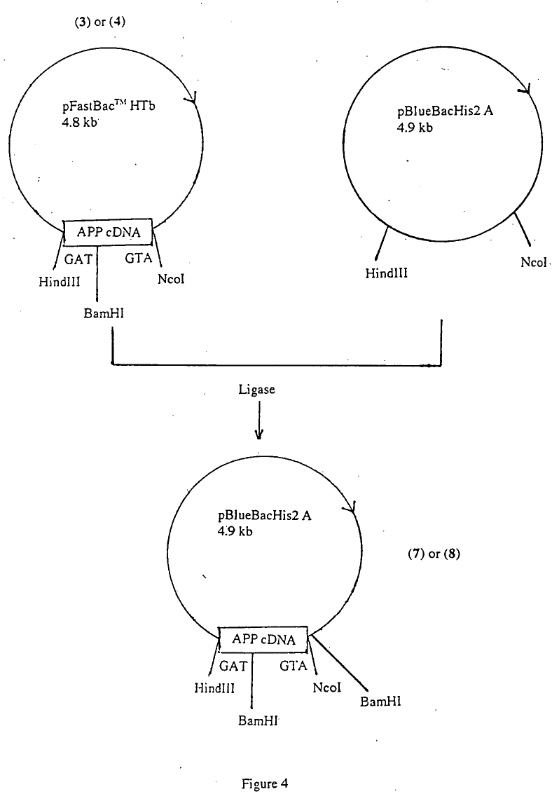

The Bac-N-Bac™ Baculovirus Expression system has been used for over a decade to produce high levels of recombinant proteins. The baculovirus transfer vector pBlueBacHis2 A (Invitrogen) is used. The pBlueBacHis2 A is a 4.9 kb polyhedrin promoter-based vector that is designed to create N-terminal fusion proteins produced in the baculovirus system. The vector's small size allows easier cloning of the gene of interest. Expression of β-galactosidase following recombination with Bac-N-Blue™ Linear DNA promotes simplified screening of recombinant plaques. Proteins expressed from pBlueBacHis2 A are fused at the N-terminus to a tag of six tandem histidine residue and an enterokinase cleavage site (the Xpress™ tag). The histidine residues create a high-affinity metal binding site to allow purification of recombinant fusion proteins on nickel-chelating resin. The Xpress™ tag is easily cleaved away from the protein using enterokinase. Expression and purification of the Xpress™ fusion protein is easily tracked using the Anti-Xpress™ Antibody which recognizes an epitope located in the Xpress™ tag. In the present work, a schematic representation of the different cloning steps for constructing the expression plasmids for APP using the pBlueBacHis2 A vector is given in FIG. 4. The correct orientation for APP expression of the APP-cDNA's insert in the plasmid vectors (7) and (8) was confirmed by the digestion of (7) and (8) with BamHI which gave two DNA fragments of 5,657 and 1,496 bp for (7) and 5,660 and 1,550 bp for (8) (data not shown).

2. Prokaryotic Expression Systems

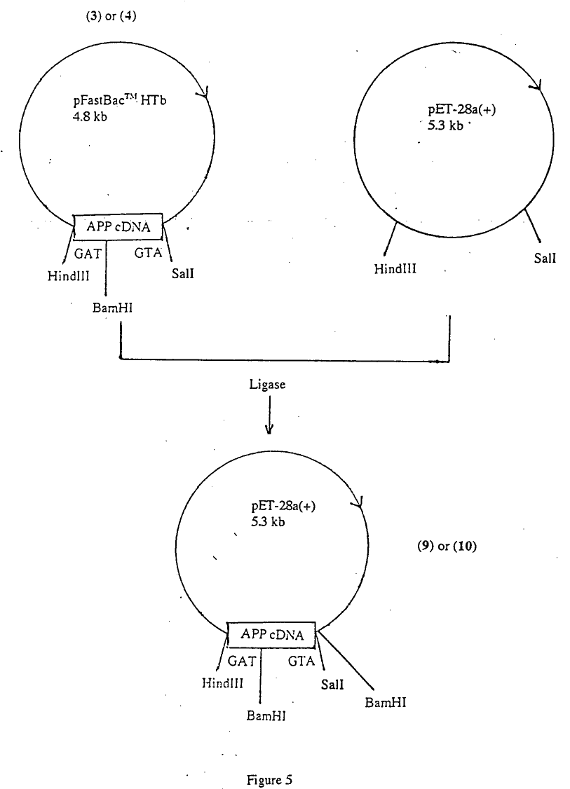

Prokaryotic hosts, especially E. Coli, have been widely used for the expression of recombinant proteins. The Prokaryotic Expression systems offer several advantages as recombinant protein expression hosts, including easy manipulation, rapid growth, and simple media requirements. Prokaryotic expression systems are well suited for expression of proteins that will be used in antibody production and for structural studies. Prokaryotic expression systems use T7 expression system which allows high-level expression from the strong bacteriophage T7 promoter and T7 RNA polymerase. The T7 expression system is ideal for expressing soluble non-toxic recombinant protein in E. Coli. In the present work, the pET-28a (+) vector (Novagen) is used; this vector of 5.3 kb carry an N-terminal His Tag/thrombin/T7 Tag configuration plus an optional C-terminal His Tag sequence. In the present work, a schematic representation of the different cloning steps for constructing the expression plasmids for APP using the pET-28a (+) vector is given in FIG. 5. The correct orientation for APP expression of the APP-cDNA's insert in the plasmid vectors (9) and (10) was confirmed by the digestion of (9) and (10) with BamHI which gave two DNA fragments of 6,057 and 1,496 bp for (9) and 6,060 and 1,550 bp for (10) (data not shown).

### CONCLUSION

The present invention is a new approach to the cloning of human βA precursor protein gene based on the RT and PCR reactions. In comparison to the old way of isolating APP-cDNA clones from the cDNA library by using synthesized oligonucleotide probes, the procedure for cloning of human APP gene via RT—PCR reactions developed herein is cost-effective, not time-consuming, and is suited for any laboratory. As a result, the construction of the expression plasmids for human APP can be performed by any laboratory. The present invention makes it easier to obtain full-length APP which is essential for the identification of biological activities that occur in the APP molecule, and for the identification of proteases capable of creating βA. Knowing which protease creates βA is important for the exploration of therapeutic and preventative strategies for the treatment of Alzheimer's disease.

### REFERENCES

1. Glenner, G. G. and Wong, C. W. Alzheimer's disease: Initial report of the purification and characterizaion of a novel cerebrovascular amyloid protein. Biochem. Biophys. Res. Commun. 120 (1984) 885-890.
2. Masters, C. L.; Simms, G.; Weinman, N. A.; Multhaup, G.; McDonalld, B. L.; Beyreuther, K. Amyloid plaque core protein in Alzheimer disease and Down syndrome. Proc. Natl. Acad. Sci. USA. 82 (1985) 4245-4249.
3. Goldgaber, D.; Lerman, M. I.; McBride, O. W.; Saffiotti, U.; Gajdusek, D. C. Characterization and chromosomal localization of a cDNA encoding brain amyloid of Alzheimer's disease. Science 235 (1987) 877-880.
4. Tanzi, R. E.; Gusella, J. F.; Watkins, P. C.; Bruns, G. A. P.; George-Hyslop, P. H. St.; Van Keuren, M. L.; Patterson, D.; Pagan, S.; Kumit, D. M.; Neve, R. L. Amyloid β protein gene: cDNA, mRNA distribution, and genetic linkage near the Alzheimer locus. Science 235 (1987) 880-884.
5. George-Hyslop, P. H. St.; Tanzi, R. E.; Polinsky, R. J.; et al. The genetic defect causing familial Alzheimer's disease map on chromosome 21. Science 235 (1987) 885-890.
6. Kang, J.; Lemaire, H. G.; Unterbeck, A.; Salbaum, J. M.; Masters, C. L.; Grzeschik, K. H.; Multhaup, G.; Beyreuther, K.; Muller-Hill, B. The precursor of Alzheimer's disease amyloid A4 protein resembles a cell-surface receptor. Nature 325 (1987) 733-736. (GenBank Accession No. Y00264).
7. Ponte, P.; Gonzalez-DeWhitt, P.; Schilling, J.; Miller, J.; Hsu, D.; Greenberg, B.; Davis, K.; Wallace, W.; Lieberburg, I.; Fuller, F.; Cordell, B. A new A4 amyloid mRNA contains a domain homologous to serine proteinase inhibitors. Nature 331 (1988) 525-527. (GenBank Accession No. Y00297).
8. Tanzi, R. E.; McClatchey, A. I.; Lamperti, E. D.; Vill-Komaroff, L.; Gusella, J. F.; Neve, R. L. Protease inhibitor domain encoded by an amyloid protein precursor mRNA associated with Alzheimer's disease. Nature 331 (1988) 528-530.
9. Kitaguchi, N.; Takahashi, Y.; Tokushima, Y.; Shiojiri, S.; Ito, H. Novel precursor of Alzheimer's disease amyloid protein shows protease inhibitory activity. Nature 331 (1988) 530-532.
10. Oltersdorf, T.; Fritz, L. C.; Schenk, D. B.: Lieberburg, I.; Johnson-Wood, K. L.; Beattie, E. C.: Ward, P. J.; Blacher, R. W.; Dovey, H. F.; Sinha, S. The secreted form of the Alzheimer's amyloid precursor protein with the Kunitz domain is protease nexin-II. Nature 341 (1989) 144-147.
11. Van Nostrand, W.; Schmaier, A. H.; Farrow, J. S.; Cunningham, D. D. Protease nexin II (amyloid β-protein precursor): a platelet α-granule protein. Science 248 (1990) 745-748.
12. Smith, R. P.; Higuchi, D. A.; Broze Jr, G. J. Platelet coagulation factor XIa-inhibitor, a form of Alzheimer amyloid precursor protein. Science 248 (1990) 1126-1128.
13. Schubert, D.; Cole, G.; Saitoh, T.; Oltersdorf, T. Amyloid beta protein precursor is a mitogen. Biochem. Biophys. Res. Commun. 162 (1989) 83-88.
14. Schubert, D.; Jin, L. W.; Saitoh, T.; Cole, G. The regulation of amyloid β protein precursor secretion and its modulatory role in cell adhesion. Neuron 3 (1989) 689-694.
15. Yankner, B. A.; Duffy, L. K.; Kirschner, D. A. Neurotrophic and neurotoxic effects of amyloid βprotein: reversal by tachykinin neuropeptides. Science 250 (1990) 279-282.
16. Whitson, J. S.; Selkoe, D. J.; Cotman, C. W. Amyloid β protein enhances the survival of hippocampal neurons in vitro. Science 243 (1989) 1488-1490.
17. Yankner, B. A.; Dawes, L. R.; Fisher, S.; Villa-Komaroff, L.; Oster-Granite, M. L.; Neve, R. L. Neurotoxicity of a fragment of amyloid precursor associated with Alzheimer's disease. Science 245 (1989) 417-420.
18. Weidemann A.; Konig, G.; Bunke, D.; Fischer, P.; Salbaum, J. M.; Masters, C. L.; Beyreuther, K. Identification, biogenesis, and localization of precursors of Alzheimer's disease A4 amyloid protein. Cell 57 (1989) 115-126.
19. Oltersdorf, T.; Ward, P. J.; Henriksson, T.; Beattie, E. C.; Neve, R.; Lieberburg, I.; Fritz, C. The Alzheimer amyloid precursor protein. Identification of a stable intermediate in the biosynthetic/degradative pathway. J. Biol. Chem. 265 (1990) 4492-4497.
20. Knops, J.; Johnson-Wood, K.; Schenk, D. B.; Sinha, S.; Lieberburg, I.; McConlogue, L. Isolation of baculovirus-derived secreted and Esch, F. S full-length β-amyloid precursor protein. J. Biol. Chem. 265 (1991) 7285-7290.
21. Esch, F. S.; Keim, P. S.; Beattie, E. C.; Blacher, R. W.; Culwell, A. R.; Oltersdorf, T.; McClure, D.; Ward, P. J. Cleavage of amyloid β peptide during constitutive processing of its precursor. Science 248 (1990) 1122-1124.
22. Suzuki, N.; Cheung, T. T.; Cai, X. D.; Odaka, A.; Otvos Jr, L.; Eckman, C.; Golde, T. E.; Younkin, S. G. An increase percentage of long amyloid β protein secreted by familial amyloid β protein precursor (βAPP₇₁₇) mutants. Science 264 (1994) 1336-1340.
23. Cai, X. D.; Golde, T. E.; Younkin, S. G. Release of excess amyloid β-protein from a mutant amyloid β-protein precursor. Science 259 (1993) 514-516.
24. Hsiao, K.; Chapman, P.; Nilsen, S.; Eckman, C.; Harigaya, Y.; Younkin, S.; Yang, F.; Cole, G. Correlative memory deficits, Aβ elevation, and amyloid plaques in transgenic mice. Science 274 (1996) 99-102.
25. Sambrook, J.; Fritsch, E. F.; Maniatis, T. Extraction, purification and analysis of messenger RNA from eukaryotic cells. In Molecular Cloning, a Laboratory Manual, 2^{nd} Ed. Cold Spring Harbor Laboratory Press: Cold Spring Harbor, N.Y. (1989) 7.28-7.52.
26. Saiki, R. K.; Scharf, S.; Faloona, F.; Mullis, K. B.; Horn, C. T.; Erlich, H. A.; Arnheim, N. Amplification of β-globine genomic sequences and restriction site analysis for diagnosis of sickle cell anemia. Science 230 (1985) 1350-1354.
27. Kawasaki, E. S.; Wang, A. M.: Detection of gene expression. In PCR Technology; Erlich, H. A., Ed. Stockton: N.Y.( 1989) 89-97.

What is claimed is:

1. The procedure for cloning human βA precursor protein gene (human APP gene) based on the reverse transcription (RT) and the polymerase chain reaction (PCR) using the synthesized oligonucleotides (SEQ ID NO. 1) for RT, and (SEQ ID NO. 2) and (SEQ ID NO. 3) respectively for PCR, comprising:
Isolating RNA.
Performing RT reaction using the synthesized oligonucleotide 5′ GTTACAGCACAG 3′ (SEQ ID NO. 1) under the following conditions: 90° C. for 2 minutes; 0° C. for 1 minute; 25° C. for 10 minutes; 42° C. for 45 minutes;
Performing PCR reaction using the synthesized oligonucleotides 5′ATGCTGCCCGGTTTGGC 3′ (SEQ ID NO. 2) and 5′CTAGTTCTGCATCTGCTCA 3′ (SEQ ID NO. 3) under the following conditions: Denaturating at 94° C. for 1 minutes; annealing at 55° C. for 2 minutes; elongating at 72° C. for 3 minutes each cycle, for 35 cycles.

2. The procedure for the construction of expression plasmids using the pFastBac™ HTb and the pBlueBacHis2 A transfer vectors for the purpose of obtaining human APP in insect cells, comprising:
2.1. Using the pFastBac™ HTb vector:
Digesting the pFastBac™ HTb vector with XbaI and HindIII followed by dephosphorylation with calf intestinal alkaline phosphatase;
Digesting the vectors (1) pCR^{R} II/APP₇₅₁-cDNA and (2) pCR^{R} II/APP₇₇₀-cDNA with XbaI and HindIII and isolating the resulting fragments containing the cDNA coding sequences of APP, APP₇₅₁-cDNA and APP₇₇₀-cDNA;
Ligating the APP₇₅₁-cDNA and APP₇₇₀-cDNA fragments to the pFastBac™ HTb vectors and introducing the ligation products in INVαF′ E. Coli strain;
Screening for inserts based on the presence of white colonies, as a result of which the vectors (3) pFastBac™ HTb/APP₇₅₁-cDNA and (4) pFastBac™ HTb/APP₇₇₀-cDNA are selected;
Introducing the vectors (3) and (4) in DH10Bac™ E. Coli competent cells;
Screening for recombinant bacmids in DH10Bac™ E. Coli using blue-white color selection, then verifying the presence of APP-cDNA's inserts in the recombinant bacmids by PCR amplification using the M13 forward (−40) and M13 reverse primers, as a result of which the recombinant bacmids (5) for vectors (3) in DH10Bac™ E. Coli and (6) for vector (4) in DH10Bac™ E. Coli respectively are selected;
2.2. Using the pBlueBacHis2 A vector:
Digesting the pBlueBacHis2 A vector with NcoI and HindIII followed by dephosphorylation with calf intestinal phosphatase;
Digesting the vectors (3) pFastBac™ HTb/APP₇₅₁-cDNA and (4) pFastBac™ HTb/APP₇₇₀-cDNA with NcoI and HindIII and isolating the resulting fragments containing the cDNA coding sequences of APP, APP₇₅₁-cDNA and APP₇₇₀-cDNA;
Ligating the APP₇₅₁-cDNA and APP₇₇₀-cDNA fragments to the pBlueBacHis2 A vectors and introducing the ligation products in INVαF′ E. Coli strain;
Screening for inserts using blue-white color selection, as a result of which the vectors (7) pBlueBacHis2 A/APP₇₅₁-cDNA and (8) pBlueBacHis2 A/APP₇₇₀-cDNA are selected.

3. The procedure for the construction of expression plasmids using the pET-28a (+) transfer vector for the purpose of obtaining human APP in bacteria, comprising:
Digesting the pET-28a (+) vector with SalI and HindIII followed by dephosphorylation with calf intestinal alkaline phosphatase;
Digesting the vector (3) pFastBac™ HTb/APP₇₅₁-cDNA and (4) pFastBac™ HTb/APP₇₇₀-cDNA with SalI and HindIII and isolating the resulting fragments containing the cDNA coding sequences of APP, APP₇₅₁-cDNA and APP₇₇₀-cDNA;
Ligating the APP₇₅₁-cDNA and APP₇₇₀-cDNA fragments to the pET-28a (+) vectors and introducing the ligation products in INVαF′ E. Coli strain;
Screening for inserts based on the presence of white colonies, as a result of which the vectors (9) pET-28a (+)/APP₇₅₁-cDNA and (10) pET-28a (+)/APP₇₇₀-cDNA are selected.
